(19) United States
(12) Patent Application Publication
(10) Pub. No.: US 2025/0288202 A1
(43) Pub. Date: Sep. 18, 2025

(54) DEVICE FOR DETECTING SUBSTANCE USE

(71) Applicant: Oculogica, New York, NY (US)

(72) Inventors: Rosina Samadani, Westport, CT (US); Daniel Sanderson, New Richmond, WI (US); Joel Sanderson, Woodbury, MN (US); David Lorek, Wilmington, NC (US); Darryl Mayeaux, Allegany, NY (US); Austin Otto, Eden Prairie, MN (US)

(73) Assignee: Oculogica, New York, NY (US)

(21) Appl. No.: 19/077,177

(22) Filed: Mar. 12, 2025

Related U.S. Application Data
(60) Provisional application No. 63/564,053, filed on Mar. 12, 2024.

(51) Int. Cl. A61B 3/113; A61B 5/11; G16H 50/20

(52) U.S. Cl. CPC A61B 3/113; A61B 5/1104; G16H 50/20; A61B 2560/0431

(57) ABSTRACT

A diagnostic device includes a main enclosure having a subject end and an operator end at least one infrared light disposed within the main enclosure adjacent the subject end, at least one stimulus lens, at least one stimulus screen disposed adjacent the operator end having a front toward a subject and a back, a camera filter disposed adjacent the stimulus screen, a mirror, a camera mount disposed adjacent the back of the stimulus screen, and at least one camera coupled to the camera mount and directed toward the mirror.

### CROSS-REFERENCE PARAGRAPH

This application claims priority to U.S. Ser. No. 63/564,053, filed Mar. 12, 2024, the content of which is hereby incorporated by reference in its entirety as if fully set forth herein.

### FIELD OF THE DISCLOSURE

The present disclosure relates to devices and systems for detecting substance use. More specifically, the disclosure relates to detecting substance abuse using eye tracking and/or pupil analysis.

### BACKGROUND

Automated eye movement tracking has been used for many purposes such as concussion diagnosis, marketing and advertising research, the development of assistive devices for immobile individuals, and for video games.

Drugs and narcotics may have an effect on eye movement. For example, methadone may impact ocular movements during both smooth pursuit and saccades, and is thought to impact function of the superior colliculus (Rothenberg et al., Psychopharmacology (Berl) 1980; 67:221-227; Rothenberg et al., Psychopharmacology (Berl) 1980; 67:229-234, 1980). Narcotic naïve subjects administered methadone had decreased smooth pursuit eye movement gain in horizontal pursuit tracking, but showed no significant decrease in gain in vertical pursuit tracking. There was a significant increase in vertical cross correlation measurements but none in horizontal cross correlation. No phase difference between subjects given methadone and control was present, signifying that the difference in gain was not due to failure of eye movement during parts of eye tracking trial or a difference in frequency of eye motion compared to target motion. The lack of vertical pursuit gain in methadone dose subjects may be due to contamination of vertical data from eyelid motion, as eyelid motion occurs with vertical eye motion when movement is greater than 5 degrees from central position. Methadone may have induced loss of eyelid control, resulting in contamination of vertical pursuit tracking. Methadone did not significantly alter maximum saccade velocity. However, initial saccade accuracy is significantly decreased with more pronounced saccade undershoot after use of methadone. In addition, the latency to onset of initial saccade was also significantly increased.

Similar results may be seen with other pharmacologic agents. Diazepam is one of the class of benzodiazepines. Subjects given diazepam showed significant decrease in smooth pursuit gain in a dose dependent manner; 5 mg diazepam significantly reduced gain at 0.4 Hz and 10 mg diazepam at 0.4, 0.6, 0.8, 1.0, 1.2, and 1.6 Hz. In contrast to methadone, diazepam induced changes in cross-correlation as function of drug as well. Phase of smooth pursuit did not show a significant change upon administration of diazepam (Rothenberg et al., Psychopharmacology (Berl) 1981; 74:232-236; Rothenberg et al., Psychopharmacology (Berl) 1981; 74:237-240). The dose dependent effects of diazepam on different frequencies of motion track suggest that smooth pursuit eye tracking after diazepam administration may be dependent on stimulus velocity. Saccadic pursuit replaces smooth pursuit upon administration of diazepam. Diazepam may induce the above eye movement changes by its binding to visual CNS benzodiazepine binding sites that are important for oculomotor control. Compared to methadone, diazepam administration shows a greater reduction in amplitude and replacement of smooth pursuit with saccadic pursuit.

Lorazepam is another of the class of benzodiazepines. When administered to subjects undergoing saccade tasks, the gap between successive images were temporally overlapped with the original image still on the screen before the next image appeared. In normal subjects, latency increases with temporal overlap compared to images separated by 200 ms gap. With lorazepam administration, subjects showed significant change during the temporal overlap but not with 200 ms gap (Masson et al., Behav Brain Res 2000; 108:169-180). Temporal overlap had no significant effect on saccadic peak velocity and amplitude in normal subjects. In lorazepam administered subjects, saccadic peak velocity and the amplitude of first saccadic eye movement significantly decreased. With smooth pursuit eye movement, lorazepam showed increased latency and longer reaction time compared to control. In addition, lorazepam significantly decreased eye velocity. Results also indicate that tracking errors in smooth pursuit induced by lorazepam are compensated for by saccadic movements of the eyes.

Alcohol consumption also impacts eye movements. Drinking subjects show decreased gain during smooth pursuit eye movement in a dose dependent manner. In one study subjects were given 0.4 and 0.8 g/kg of alcohol and eye tracking was done on two time points: T1 at 60 min. and T2 at 180 min. after beverage consumption (Roche et al., Psychopharmacology (Berl) 2010; 212:33-44). In smooth pursuit eye tracking, high dose affected gain at both time points while low dose did not have an effect on gain for the latter time point. For pro-saccade, latency was also impaired in a similar, dose dependent manner. Ocular velocity and accuracy decreased only after high dose consumption. Anti-saccade showed similar presentation as pro-saccade with the exception that high dose improved accuracy at T1 and decreased by T2.

Alcohol significantly affected both pro and anti-saccade accuracy; however, greater accuracy for high dose alcohol at T1 may be due to alcohol increasing the amplitude of anti-saccade relative to normal conditions and not that alcohol is improving anti-saccade functioning. This suggests that high dose alcohol may be affecting neurocircuitry required for rapid processing of visuospatial information. High dose and low dose alcohol consumption show similar impairment in smooth pursuit gain and anti-saccade functions; however, high dose patients have less awareness of the impact of this dysfunction, placing them in higher risk for injuries.

It would be highly advantageous to continue to improve methods and systems for detecting substance use or abuse. It would also be ideal to be able to assess, quantify or analyze the severity of inebriation or intoxication or impairment. At least some of these objectives will be discussed in the present application.

### BRIEF SUMMARY

In some embodiments, a diagnostic device includes a main enclosure having a subject end and an operator end, at least one infrared light disposed within the main enclosure adjacent the subject end, a stimulus lens, a stimulus screen disposed adjacent the operator end having a front toward the user and a back, a stimulus filter disposed adjacent the stimulus screen, a mirror, a camera mount disposed adjacent the back of the stimulus screen, and a camera coupled to the camera mount and directed toward the mirror.

In some embodiments, a diagnostic device includes a main enclosure having a subject end and an operator end, at least one infrared light disposed within the main enclosure adjacent the subject end, a stimulus lens, a stimulus screen disposed adjacent the operator end having a front toward the user and a back, a stimulus filter disposed adjacent the stimulus screen, a mirror, a camera mount disposed adjacent the back of the stimulus screen, a camera coupled to the camera mount and directed toward the mirror, a power source disposed to one side of the main enclosure, and a printed circuit board disposed within the main enclosure opposite the power source, wherein the main enclosure has a center of mass that is closer to the operator end than the subject end.

In some embodiments, a method of diagnosing a subject includes providing diagnostic device including a main enclosure having a subject end and an operator end, at least one infrared light disposed within the main enclosure adjacent the subject end, a stimulus lens, a stimulus screen disposed adjacent the operator end having a front toward the user and a back, a stimulus filter disposed adjacent the stimulus screen, a mirror, a camera mount disposed adjacent the back of the stimulus screen, a camera coupled to the camera mount and directed toward the mirror, a power source disposed to one side of the main enclosure, a printed circuit board disposed within the main enclosure opposite the power source, and an operator display disposed on the operator end, holding the diagnostic device so that the operator display faces an operator and the stimulus screen faces the subject, and providing one or more stimuli on the stimulus screen and presenting results to the operator on the operator display.

### BRIEF DESCRIPTION OF THE DRAWINGS

FIG. 1 is a schematic diagram illustrating a system for diagnosing, identifying and/or quantifying substance abuse in a subject, according to one embodiment.

FIG. 2 is a schematic flow chart illustrating a method for diagnosing, identifying and/or quantifying substance abuse in a subject, according to one embodiment.

FIG. 3A is a schematic flow chart illustrating a predictive method for identifying substance abuse in a subject, according to one embodiment.

FIG. 3B is a schematic flow chart illustrating a predictive method for identifying substance abuse in a subject, according to one embodiment.

FIGS. 4A-4J illustrate several views of a diagnostic device, according to one embodiment.

FIGS. 5A-5G illustrate several views of the internal components of the diagnostic device shown in FIGS. 4A-4J.

### DETAILED DESCRIPTION

As used herein, the term “narcotic” refers to any psychoactive compound with any sleep-inducing properties, or any drug that is prohibited, such as heroin or morphine. It is meant to include, for instance, opiates, opioids, morphine, heroin and their derivatives, such as hydrocodone as well as cannabis, alcohol, and any other substance classified as a narcotic by the United States Controlled Substances Act. In some instances, the drug or narcotic may be a prescription medication such as, for instance, a benzodiazepine or barbiturate.

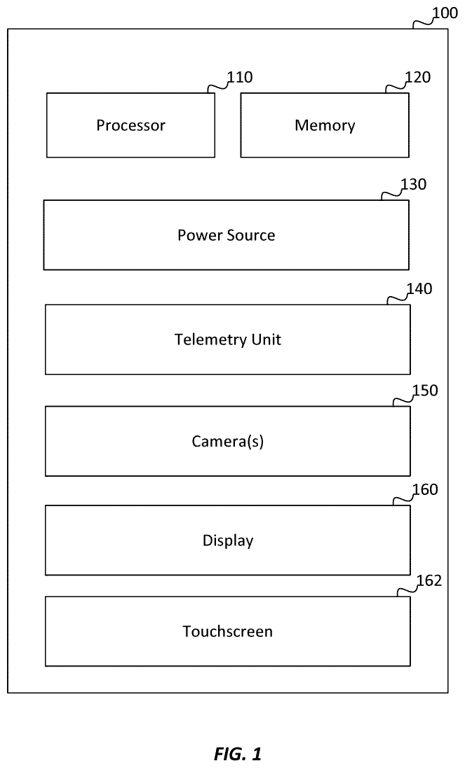

Referring to FIG. 1, a schematic of an eye tracking or monitoring, biometric identification and/or diagnostic device 100 is illustrated, according to one embodiment. The eye tracking device 100 may take the form of a portable, handheld device, and the eye tracking system may be used to track a patient's eye movement and diagnose one or more eye movement abnormalities. It will be understood that the eye tracking device can include more, fewer, or different components and can have a variety of different configurations. Additionally, some of the components may be positioned on one or more circuit boards or similar carriers.

Generally, eye tracking device 100 may include a one or more processors 110, a memory 120 (e.g., microSD card adapter), a power source 130, a telemetry unit 140, camera(s) 150 and a display 160. Any of these components may be optional. Processor 110 may include a single microcontroller, or divided amongst more than two microcontrollers. In this example, a processor 110 is included to generate a stimulus and obtain data from the data capture device, for example, a camera 150. It will be understood that other parameters and sensors may be used to capture additional data from the user. Any processor can be used and can be as simple as an electronic device that, for example, is capable of receiving and interpreting instructions from an external programming unit and performing calculations based on the various algorithms described above.

A memory 120 may include data in the form of a dataset for performing various steps of the algorithm. For example, in some examples, data from camera 150 regarding a characteristic of the eyes may be passed to the processor 110 and compared against a dataset stored in memory 120 to determine if further diagnosis is necessary. Additionally, data relating to characteristic of the eyes may be sent from a programming unit to processor 110 and the processor may determine the appropriate course of action or alert a user and/or clinician. Communication between a programming unit and processor 110 may be accomplished via communication of an antenna with telemetry unit 140. Additionally, device 100 may be in communication with one or more wearable devices or external devices to enable the user to continuously monitor or track the data. Telemetry unit 140 may be capable of transmitting data from memory 120 to a server or a network. As discussed, the system may include a single microcontroller. Additionally, systems having one or more microcontrollers may include a single power source, or a single display 160 (e.g., a touchscreen LCD that can also function as a data input device).

Any power source 130 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like. If the power source 130 is a rechargeable battery, the battery may be recharged using an antenna of a telemetry unit 140, if desired. Power can be provided to the battery for recharging by inductively coupling the battery through the antenna to a recharging unit external to the user.

In one particular configuration, camera 150 may include a machine vision camera or “smart camera(s)” that to capture visual information from the surrounding environment, under potentially challenging lighting conditions, and provide high-resolution images with precise color accuracy and optimal resolution. Eye tracking device 100 may also include a liquid lens auto-focus lens, and an ARM-based computer (SoM/System-on-Module form factor), with Wi-Fi and Bluetooth. The power source may be a lithium-ion rechargeable battery. A display 160 in the form of a 3.5″ stimulus screen may be disposed inside the device, and an external 3.0″ touchscreen 162 (which may also be referred to as an “operator console” or simply “touchscreen”) to be used by the technician to interact with the system application. Touchscreen interface 162 may include only a touch screen display, meaning that there is no keyboard or other input device. Of course, alternative embodiments may include a keyboard or other input device(s). Eye tracking device 100 may also include one or more IR LED illuminators disposed adjacent the eyes, and a stimulus lens by the eyes, which may be in the form of either Fresnel or spherical. In some examples, the stimulus lenses may be acrylic. Optionally, eye tracking device 100 may include speakers (e.g., on the bottom), a power button, a USB-C charging port, a USB-A (2.0) data port, LED power and charging indicators, a durable housing including drop-resistant materials, especially for the side handles, a tripod mount on the bottom, and an eye-piece soft material that conforms to a person's face to block out light.

### Eye Tracking Computer

In one embodiment, camera 150 may be driven by an ARM-based embedded computer. The specifications for this eye tracking computer are shown in Table 2.

TABLE 2
NXP i.MX 8 System on Module

Spec Description
Design System on Module (SOM)
PCB size: 40 mm × 47 mm
Embedded CPU Quad-core ARM A53 @ 1.6 GHz
Connectivity WiFi and Bluetooth

### Stimulus Display

A camera computer, which may be provided by the same manufacturer as the manufacturer of camera(s) 150, may run the real-time software for camera 150. It detects eye motion events, such as saccades, blinks, and fixations, and computes the gaze coordinates for each eye at 30 to 500 Hz, storing the raw data until it is needed by the application. The application computer may be a small form-factor PC that runs a system application for the system. The system application provides the user interface, controls the logic flow, displays the stimulus video, processes the raw data from the camera computer, and stores results in persistent storage.

The user interacts with the system application through one or more displays having physical buttons or keys, or touchscreen interface(s) 160. Displays 160 may provide stimulus media to the patient, and may include speakers to provide the audio for the stimulus media.

Display 160, according to one embodiment, is used to present a video that may last any suitable length of time, such as 220 seconds in one embodiment. In one embodiment, the only purpose of a stimulus screen is to display the visual stimulus and the terms “display” and “stimulus screen” are synonymous. The video may be one of several predetermined videos, visual patterns, or light-emitting devices. The videos may include music videos, clips from children's movies, sports clips, talent performances, “reality TV” clips, etc. The choice of videos may be designed to appeal to a broad group of subjects. Users of the device may choose which video to display or may ask the patient which one they would like to watch. The visual patterns may include geometric or natural shapes or designs, moving or not moving. The light-emitting devices may include LEDs or fluorescent illumination devices. In one embodiment, stimulus screen is a 3.5″ TFT LCD Display, with the specifications shown below in Table 4.

TABLE 4
Stimulus Screen Specifications

Spec Description
Aspect ratio 1.66:1
Maximum resolution 800 × 480
Screen size 3.5 inches
LCD TFT
Viewing angle 89°/89°/89°/89°

### Touchscreen Interface

The system may include a designated touchscreen interface 162 (which may also be referred to as an “operator console” or simply “touchscreen”) to be used by the technician to interact with the system application. Touchscreen interface 162 may include only a touch screen display, meaning that there is no keyboard or other input device. Of course, alternative embodiments may include a keyboard or other input device(s). In one embodiment, touchscreen interface 162 may be a 3″ TFT LCD Display, with the specifications set forth below in Table 5. Alternatively, touchscreen 162 and display 160 may be the same component.

TABLE 5
Touchscreen Interface Specifications

Spec Description
Aspect ratio 1.78:1
Maximum resolution 854 × 480
Screen size 3.0 inches
LCD TFT
Viewing angle 89°/89°/89°/89°

### Substance Abuse Detection Method

In some embodiments, a substance abuse detection method 200 may include a series of steps for collecting and analyzing pupil size and position data from a subject using a hand-held, battery-powered device equipped with an infrared camera similar to that described above. The subject may be instructed to look into the device (or at a display of the device) and to observe a stimulus that may consist of pseudorandom changes in illumination and/or images that the subject follows (step 210). Optical lenses may be used to make the focal length appropriate for most subjects (about 10-40 cm). The optical lens may be a Fresnel lens or spherical lens. In some examples, pseudorandom changes may include changing the illumination from complete darkness to maximum brightness), or vice versa, in various steps or intervals (e.g., 5%, 10%, 25% intervals). For example, an illumination pattern may consist of presenting the following levels of illumination: 5%, 85%, 45%, 70%, 40%, 20%, 85%, etc. Such changes may evoke pupil constriction or dilation, and may occur in either (or both) direction (i.e., increasing in illumination or decreasing in illumination). In some examples, a square or sawtooth wave pattern may also be implemented.

If images are used, the image may move in any direction (e.g., left, right, up or down) in pseudorandom patterns or remain fixed on the screen in one position. In some examples, the movements of the image may be abrupt. In some other examples, the movements of the image may be smooth. Alternatively, the movements may include any combination of smooth and abrupt movements or fixed positioning. A naïve subject will not be able to predict the changes in illumination or image movement. In some examples, the image may evoke interest from the subject. For example, the image may be a football, flower, car, or other common object, and may change during the stimulus. The stimulus may be presented for 5 seconds, 10 seconds, 20 seconds, 30 seconds, 45 seconds, 1 minute or 1:30 seconds or 2 minutes in total.

In some embodiments, a substance abuse detection method 200 may include a stimulus pattern in which light is removed from the subject's view. This dark period may be presented for 5 seconds, 10 seconds, 20 seconds, 30 seconds, 45 seconds, 1 minute or 1:30 seconds, or 2 minutes in total.

During the stimulus presentation, an infra-red camera(s) captures the images of the subject's eyes and calculates each pupil size and/or gaze position and stores them in the device memory (step 220). In some examples, a designated series may be assigned to each of the subject's eyes for pupil size and/or gaze (e.g., position data).

After the stimulus is complete, the device may filter the captured pupil size and/or position data to remove noise (step 230). The data may be normalized and adjusted for demographic information, such as age or gender (step 240). For example, it is known that pupil sizes in older normal adults are usually smaller than in younger normal adults.

Various metrics may be derived from the data. As one example, a series may be divided into overlapping vectors with a maximum size of 500 frames (step 250). The vectors may overlap with each other by 50 frames, e.g., the first vector may contain pupil sizes 1 to 500, the second vector may contain pupil sizes 51 to 551, and so on. Missing values may be ignored. Each vector may be transformed into frequency data using Fourier transformation and the magnitudes of each frequency may be computed by taking the square of the sum of the squares of the real and imaginary portion of each complex number in the Fourier transform result (step 260). These magnitudes are accumulated across all vectors, and the sum of the accumulated magnitudes may be computed (step 270). This value of the sum of the accumulated magnitudes may become one of the inputs to a prediction algorithm (e.g., via logistic regression) (step 280). Alternatively, the vector data may be transformed into frequency data by using low-pass, band-pass, and/or high-pass filters.

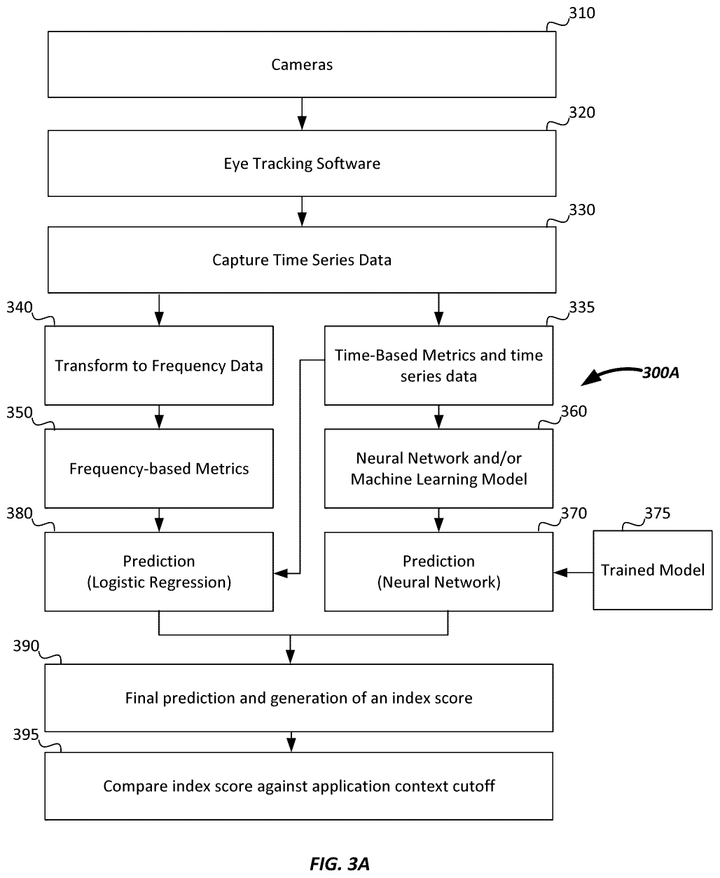

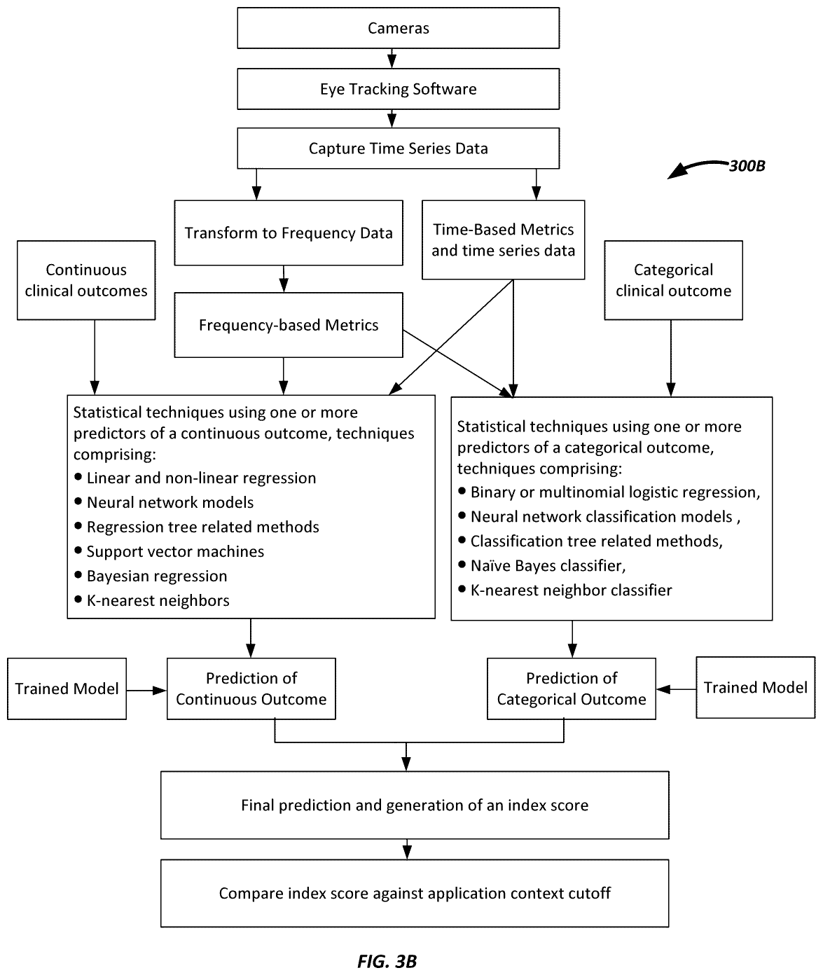

A predictive algorithm 300A and the flow of data is shown in FIG. 3A. Cameras 310 may capture data and send it to eye tracking software 320. Time series data may be captured 330 and time-based metrics and time series data may be calculated 335. Time series data may be transformed to frequency data 340 to calculate frequency-based metrics 350. As shown, in addition to the frequency data, the time series data may also be analyzed for various characteristics, such as average, median, and/or variance of gaze positions and/or pupil sizes. These values may also become inputs to a prediction algorithm (e.g., via a neural network 360).

In some examples, the time series data (pupil size changes and/or gaze position changes) may also be submitted to a prediction engine 370 that was trained using a Convolutional Neural Network. In this case, data from normal and impaired subjects are collected in a controlled study. A trained model 375 is built from these data and is used to predict impairment using the data from a person who is suspected of impairment.

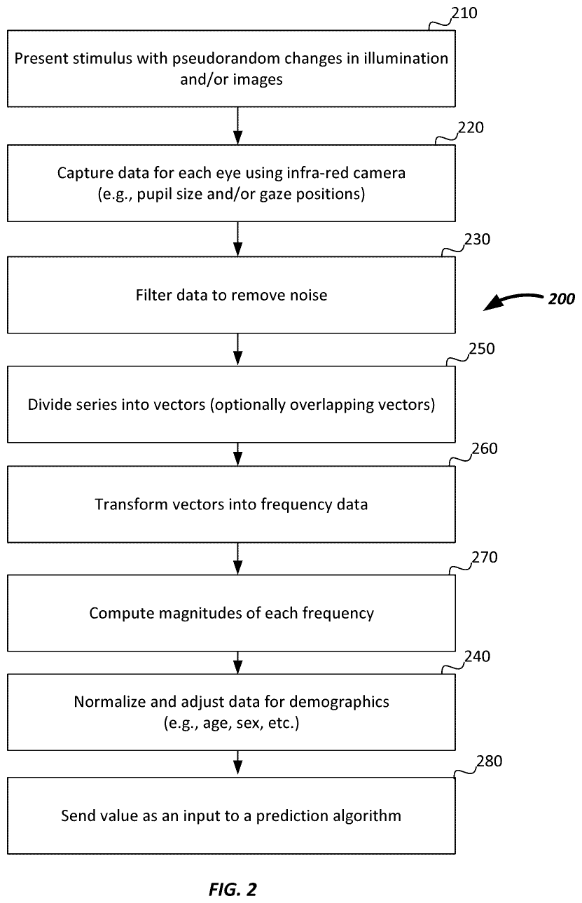

The output of the prediction algorithm 380 from time-series-based data and the output of the Neural Network prediction 370 may be used together to establish a final prediction 390 using a linear or polynomial formula to convert the two outputs into a final prediction with a composite index score 395 that can range from 0 to 100 where a 0-index score means unlikely to be impaired and a 100-index score reflects a high probability or likelihood of the subject being impaired. A predetermined or predefined application context cutoff that determines impairment may be established depending on where and how the test will be applied. For example, in law enforcement, the cutoff of the index score may be higher to avoid false positives and possible false arrests (e.g., an index score of equal to or greater than 90 may signal a strong likelihood of impairment). In a manufacturing environment, the cutoff may be lower to avoid accidents (e.g., index score of equal to or greater than 50 may signal a sufficient likelihood of impairment so as to remove an employee or worker from a potentially dangerous environment). It will be understood that similar analysis may also be conducted to ensure that an individual is fit for a given task (e.g., that the individual is not sleep-deprived, intoxicated, tired, etc.). The outlined methods of FIGS. 2 and 3 are exemplary, and variations may be made without departing from the scope of the disclosure. For example, alternative embodiments may include fewer steps, greater numbers of steps and/or different ordering of steps.

In one variation, shown in FIG. 3B, other statistical techniques may be used in a predictive algorithm 300B. For example, continuous clinical outcomes and categorical clinical outcomes may be collected. In some examples, the continuous clinical outcomes may be used in statistical techniques using one or more predictors of a continuous outcome, and the techniques may include (but are not limited to) one or more of linear and non-linear regression, neural network models, regression tree related methods, support vector machines, Bayesian regression, and K-nearest neighbors. In some examples, categorical clinical outcomes may be collected and statistical techniques using one or more predictors of a categorical outcome, may include one or more of binary or multinomial logistic regression, neural network classification models, classification tree related methods, Naïve Bayes classifier, and K-nearest neighbor classifier. In this example, linear regression may be used and the outcome may be a continuous outcome, as opposed to the categorical outcomes for logistic regression. Predictions of both continuous and categorical outcomes may be performed and both may be used to create a final prediction and generate an index score with predetermined cutoffs as previously described.

In another variation, neither statistical nor machine learning techniques may be used to develop an algorithm. Instead, one or more cutoffs for any kind of eye metric may be established to differentiate two or more clinical classifications. The classification from one kind of metric may be combined with the classification from one or more additional metrics to produce a final score predicting clinical classification.

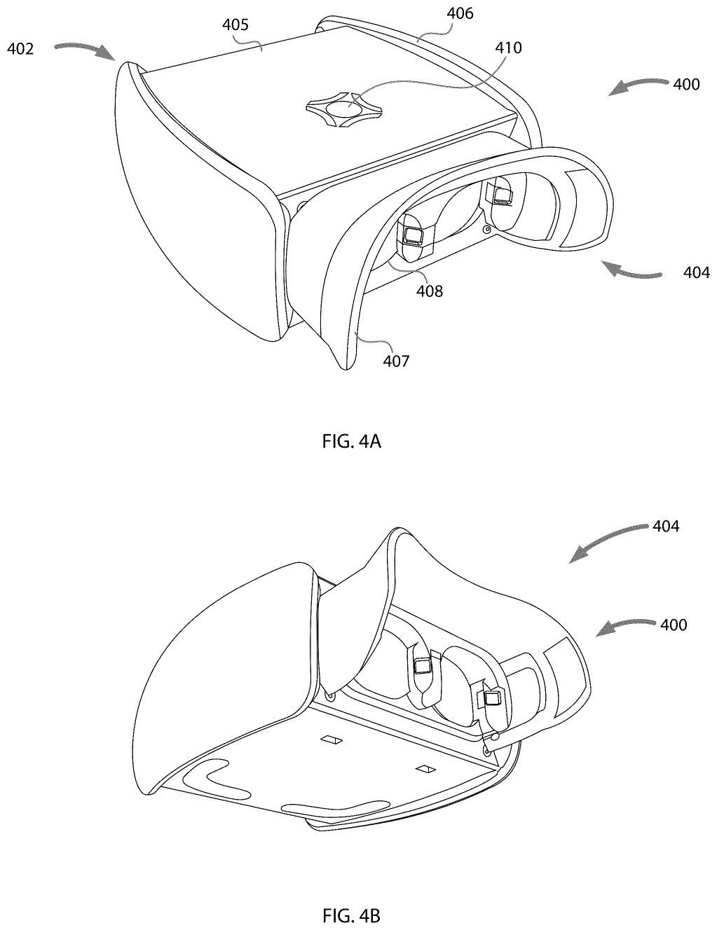

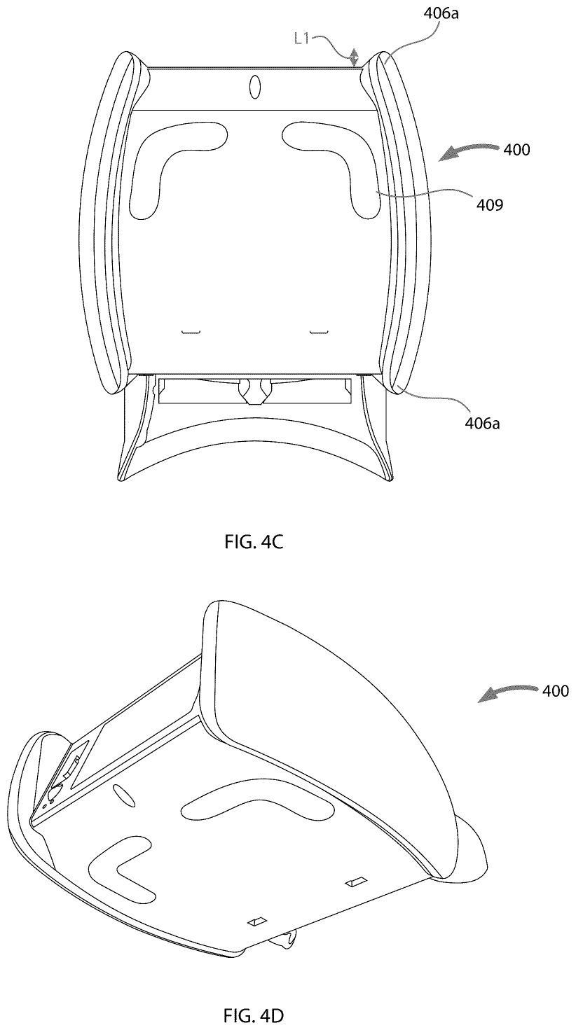

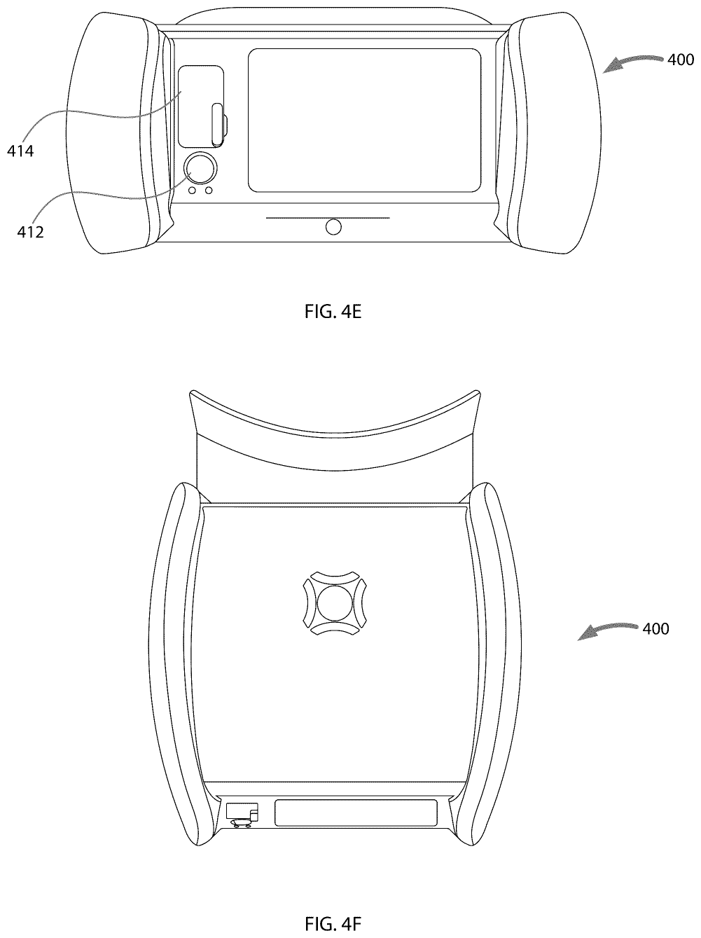

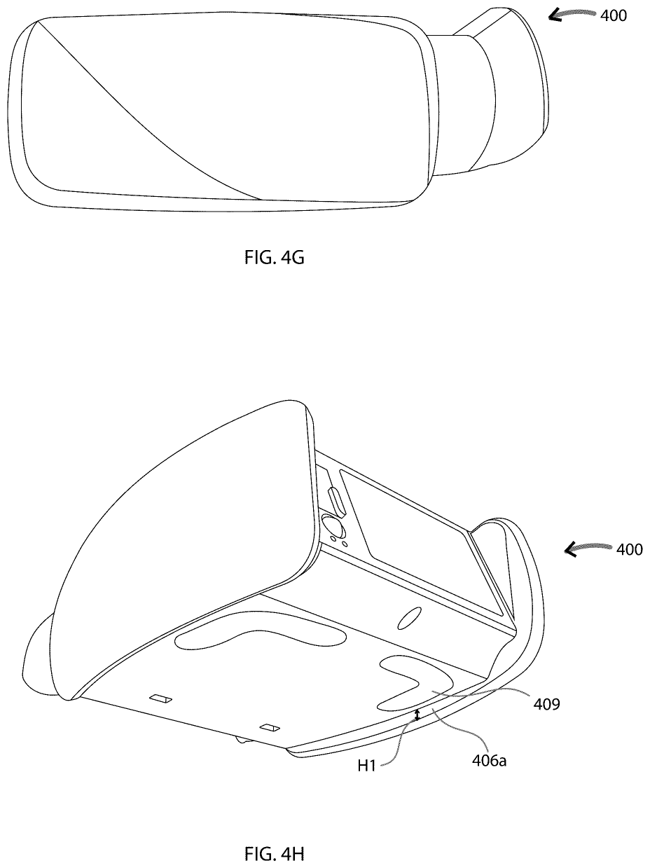

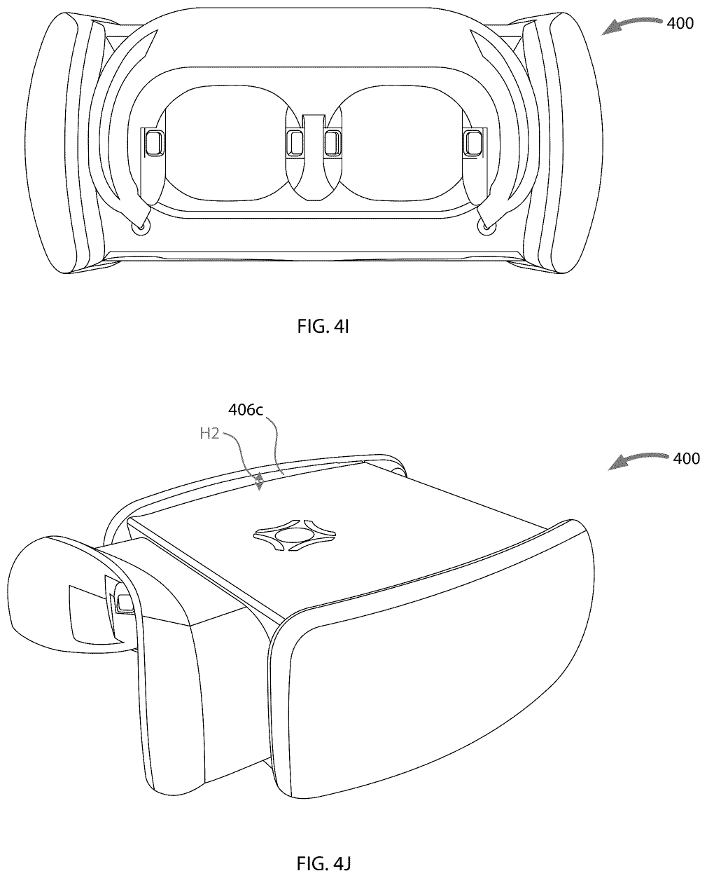

Disclosed herein are certain devices that may be used to detect substance use, intoxication, drowsiness, abnormal eye movement, concussions, brain damage, dyslexia, ADHD, autism and/or other neurological conditions. FIGS. 4A-J show certain views of a diagnostic device 400, according to one embodiment. Diagnostic device 400 may take the form of a portable, handheld device, and the eye tracking system may be used to track a patient's eye movement or pupillary changes and diagnose one or more eye movement or eye response abnormalities. It will be understood that the eye tracking device can include more, fewer, or different components and can have a variety of different configurations. Additionally, some of the components may be positioned on one or more circuit boards or similar carriers.

As shown in FIG. 4A, diagnostic device 400 may extend between an operator end 402 and an opposing subject end 404. It will be understood in this context, that the operator and the subject may be different persons. That is, the operator may be a law enforcement agent, medical personnel, supervisor, or other diagnosing personnel that holds and conducts the test on a subject. To aid in carrying diagnostic device 400, main enclosure 405 may be provided with opposing indentations 409 on the bottom surface, where the operator may place their digits (e.g., thumbs). In some examples, indentations 409 are generally L-shaped and oriented to allow the operator to hold the device in multiple positions. In some examples, the operator may provide the subject with instructions on how to use the device. In this embodiment, diagnostic device 400 includes a main enclosure 405 configured to house the hardware of the diagnostic device 400 (e.g., camera, processor, lenses, etc.), and a pair of handles 406 disposed on opposite sides of the main enclosure. As best shown in FIGS. 4C, 4D, 4F and 4H, handles 406 may be formed of a durable, drop-resistant, shock-proof or shock-absorbent material (e.g., hard plastics or rubber) to protect main enclosure 405. In some examples, handles 406 may be oversized and have terminal lips 406a that extend proximally and distally past main enclosure 405 by an offset distance L1 so that they would be impacted first in case of a drop. In some examples, the offset distance is between ¼ inch and 3 inches (FIG. 4C). In some examples, handles 406 may be taller than main enclosure 405 and may have upper and lower lips 406b,406c that extend past main enclosure 405 by an offset distance H1,H2 so that they would be impacted first in case of a drop (FIGS. 4H and 4J). In some examples, the offset distance H1,H2 is between ¼ inch and 3 inches. In some examples, offset distances H1,H2 are equal. In some examples, offset distances H1,H2 are unequal and H1 may be greater than H2, or vice versa. Thus, handles 406 may have dimensions that are larger than main enclosure 405 to partially surround all edges of the main enclosure to protect it from damage. In other words, each of the pair of handles may be offset and extend farther outwardly from each of a top surface, a bottom surface, a front surface and a back surface of the main enclosure

At the subject end 404, the device may include a removable visor 407 configured to cover or enclose both eyes and at least a portion of a face of the subject when held to the subject's eyes. In some examples, visor 407 may function as an opaque shield to regulate light conditions by effectively blocking out ambient light, thereby creating a state of complete or near darkness for the eyes. Visor 407 may be constructed from a thin, rigid material such as plastic, rubber, rubberized silicone or hard plastic. In some examples, visor 407 is releasable from the main enclosure 405, and it may be made in different sizes to accommodate different subjects. Additionally, visor 407 may be replaced after each use for safety of the subject or may include a disposable covering. One or more windows 408 are shown in FIG. 4A, where the subject will look into the main enclosure 405.

Optionally, main enclosure 405 may include an ambient light sensor 410 configured to measure the amount of ambient light in the device's surroundings. Ambient light sensor 410 may work by detecting the intensity of the environmental light and may be used as a variable in the algorithms described above. As ambient light conditions change, sensor 410 may also automatically adjust the brightness of the display to make it more readable in varying lighting environments. For example, in bright outdoor settings, the display brightness may increase to improve visibility, while in dimly lit settings, the brightness may decrease to reduce eye strain and conserve battery power. At the subject end 404, the main enclosure may also include a power connection 412 and a USB connection 414 for storing data and/or communicating with external devices (FIG. 4E).

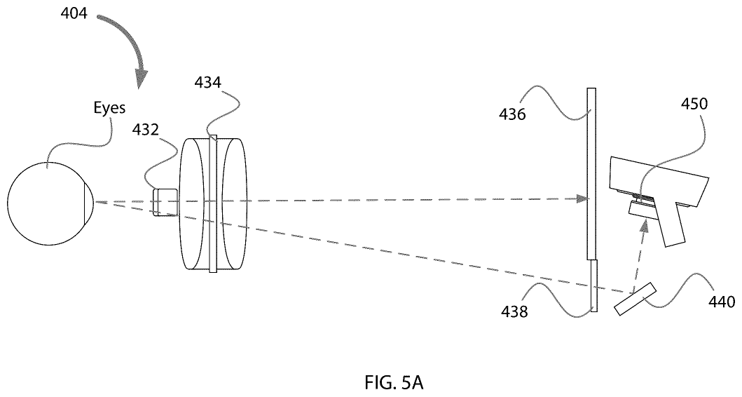

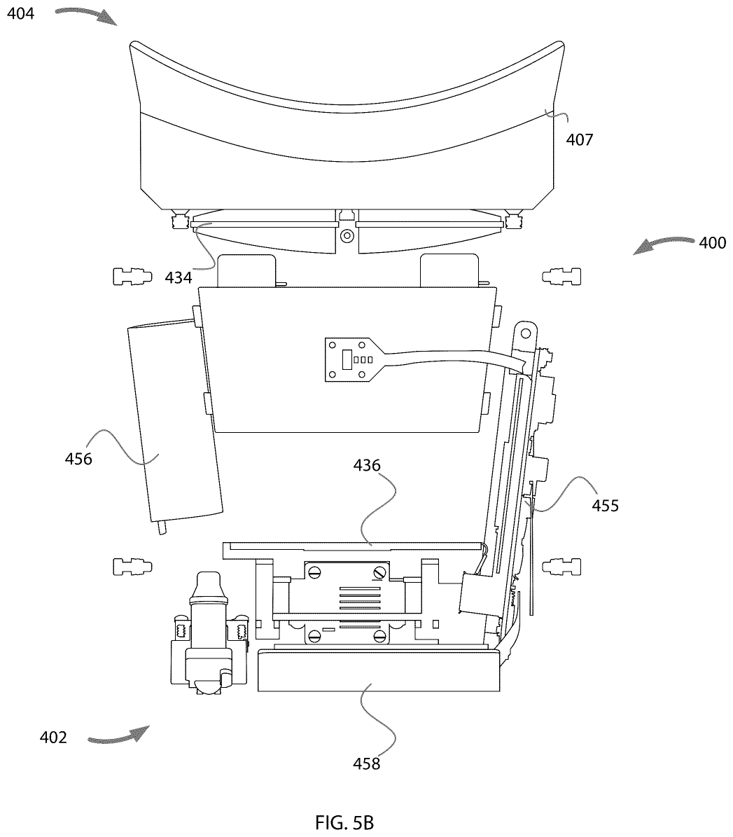

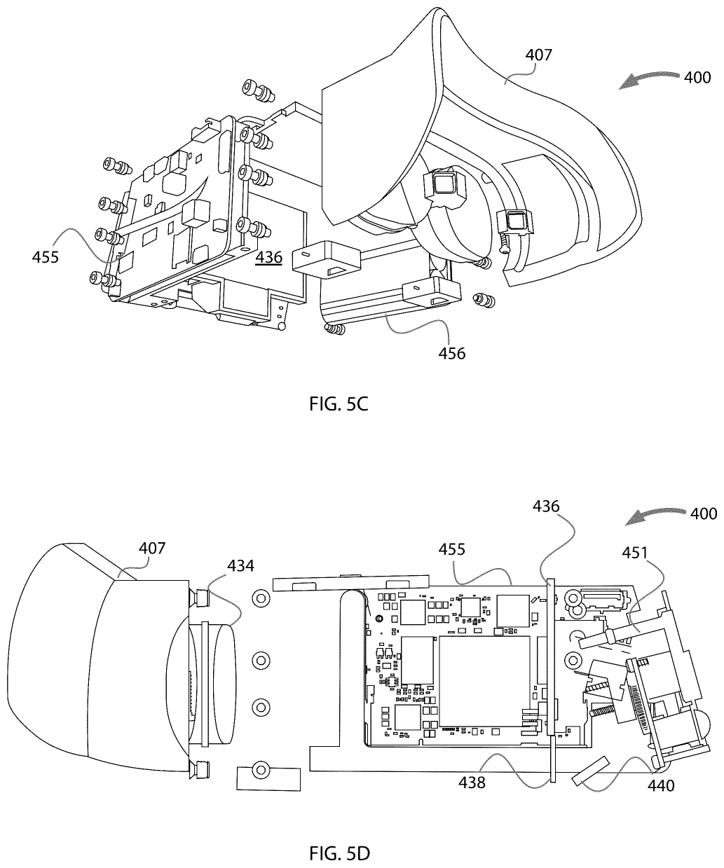

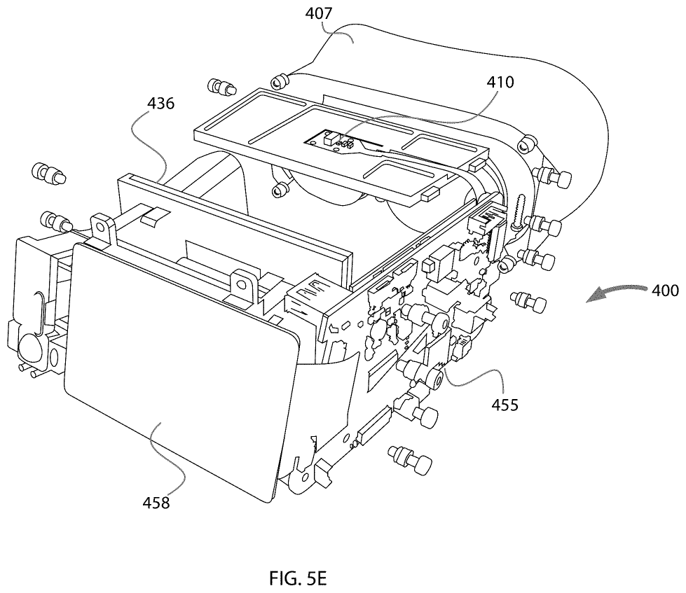

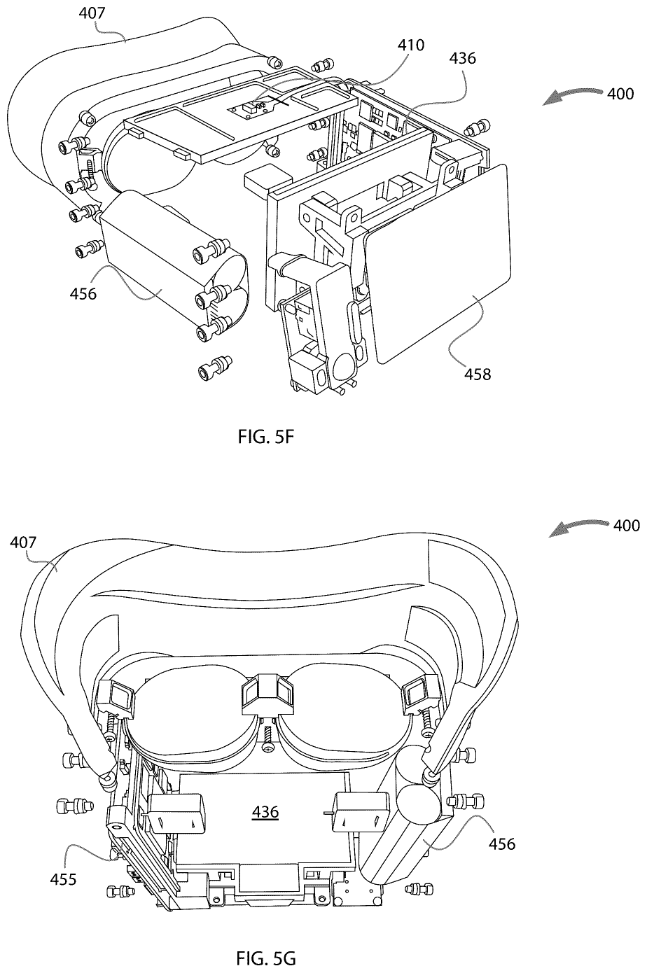

Inside main enclosure 405, diagnostic device 400 may include any of the components described above with reference to FIG. 1. In one particular arrangement shown in FIGS. 5A-5G, the main enclosure and other components are not shown and certain features are isolated for clarity of explanation. In this example, it will be seen that eyes of the subject will be disposed adjacent subject end 404. Moving from the subject end 404 toward the operator end 402, diagnostic device 400 may include one or more IR light(s) 432 (e.g., infrared LED illuminators angled by 45 degrees toward each eye), a stimulus lens 434, one or more stimulus screen(s) 436 (e.g., a two-piece screen configured to form a single optical image), an IR-passing filter 438 configured to block visible light and allow infrared light to pass therethrough, a first surface mirror 440 and a camera 450. Camera 450 may include a liquid lens auto-focus lens. To aid in understanding the spacing of these elements, each square shown in FIG. 5A represents approximately one centimeter. The subject may look at stimulus screen 436 to observe an image, pattern or change in illumination as described above, and IR light 432 may be directed at the eyes. Eyes of the subject may be positioned to look at stimulus screen 436. Meanwhile, stimulus lens 434, IR-passing filter 438, mirror 440 and camera 450 may be positioned so that camera 450 can track, analyze and record changes in the eyes in response to a stimulus from stimulus screen 436. In some examples, camera 450 is oriented toward mirror 440 via mount 451 (FIG. 5D). In some examples, mirror 440, camera 450, and camera mount 451 may be disposed in other positions (e.g., next to the stimulus screen or in front of the stimulus screen).

Diagnostic device 400 may include a circuit board having one or more processors 455 disposed on one side of the device and a power source in the form of a rechargeable battery 456 disposed on the other side of the device opposite processors 455. In some examples, processor(s) 455 and/or rechargeable battery 456 may be positioned closer to operator 402 than subject end 404. In some examples, the position of processor 455, rechargeable battery 456 and other components (e.g., stimulus screen 436, the camera, the display screen, etc.) results in the device having a center of mass that is closer to operator end 402 than subject end 404. This particular configuration is intentional as the device is configured to be carried up and held up by an operator to a subject and moving the center of mass closer to the operator makes the device more stable in the hands of the operator.

As shown in FIG. 5B, diagnostic device 400 also includes an operator display screen 458 that is oriented toward the operator and configured to provide information to the operator. In some examples, display screen 458 is slanted or upwardly inclined to accommodate easy usage and viewing of the screen, for example at 15 to 20 degrees from vertical. In some examples, display screen 458 is configured to show a real-time video (or images) of the subject's eyes during the diagnostic test. In other examples, display screen 458 is configured to show information, graphs, data, and/or statistics relating to the test. In some examples, display screen 458 is configured to show images, video or data relating to pupillary dilation rebound. The video and/or information may also be recorded (e.g., .mp4 video recording of the eyes for later manual review) and stored on a memory or sent to another device, or the cloud, via a telemetry unit or exported to removable storage. The data may be stored for later review, for archiving purposes and/or to perform aggregated data analytics.

In some embodiments, a hand-held diagnostic device is provided that uses eye-tracking technology or eye analysis and a data processing algorithm to detect subtle changes in eye movements or changes resulting from concussion, intoxication, inebriation, substance abuse, or impairment. The hand-held diagnostic device may diagnose a subject in about 90 seconds using any of the algorithms previously described. In addition to changes in lighting or patterns, a video may also move around the perimeter of the stimulus screen while sensors in the visor-shaped device record eye movements. The data is then analyzed to produce one or more outcome measures. In some examples, diagnostic device 400 may include software that allows for automatic pupil finding or recognition.

The foregoing is believed to be a complete and accurate description of various embodiments of a system and method for assessing substance abuse in a person. The description is of embodiments only, however, and is not meant to limit the scope of the invention set forth in the claims.

1. A diagnostic device comprising:
a main enclosure having a subject end and an operator end;
at least one infrared light disposed within the main enclosure adjacent the subject end;
at least one stimulus lens;
at least one stimulus screen disposed adjacent the operator end having a front toward a subject and a back;
a camera filter disposed adjacent the at least one stimulus screen;
a mirror;
a camera mount disposed adjacent the back of the at least one stimulus screen; and
at least one camera coupled to the camera mount and directed toward the mirror.

2. The diagnostic device of claim 1, further comprising an operator display screen disposed adjacent the operator end.

3. The diagnostic device of claim 2, wherein the operator display screen is inclined.

4. The diagnostic device of claim 1, further comprising a visor releasably coupleable to the subject end of the main enclosure.

5. The diagnostic device of claim 1, further comprising a pair of handles disposed on sides of the main enclosure.

6. The diagnostic device of claim 5, wherein each of the pair of handles is offset and extends outwardly farther from each of a top surface, a bottom surface, a front surface and a back surface of the main enclosure.

7. The diagnostic device of claim 1, wherein the main enclosure has a bottom surface that includes two indentations disposed closer to the operator end.

8. The diagnostic device of claim 1, further comprising a power source.

9. The diagnostic device of claim 8, wherein the power source is a rechargeable battery disposed to one side of the main enclosure.

10. The diagnostic device of claim 9, wherein the rechargeable battery is disposed closer to the operator end than the subject end.

11. The diagnostic device of claim 9, further comprising a printed circuit board disposed within the main enclosure opposite the rechargeable battery.

12. The diagnostic device of claim 11, wherein the printed circuit board is disposed closer to the operator end than the subject end.

13. The diagnostic device of claim 1, further comprising an ambient light sensor disposed on an upper surface of the main enclosure.

14. A diagnostic device comprising:
a main enclosure having a subject end and an operator end;
at least one infrared light disposed within the main enclosure adjacent the subject end;
a stimulus lens;
a stimulus screen disposed adjacent the operator end having a front toward a subject and a back;
a camera filter disposed adjacent the stimulus screen;
a mirror;
a camera mount disposed adjacent the back of the stimulus screen;
a camera coupled to the camera mount and directed toward the mirror;
a power source disposed to one side of the main enclosure; and
a printed circuit board disposed within the main enclosure opposite the power source;
wherein the main enclosure has a center of mass that is closer to the operator end than the subject end.

15. The diagnostic device of claim 14, further comprising an operator display screen disposed adjacent the operator end.

16. The diagnostic device of claim 15, wherein the operator display screen is inclined.

17. The diagnostic device of claim 14, further comprising a visor attached to the subject end of the main enclosure.

18. The diagnostic device of claim 14, further comprising a disposable covering releasably coupled to the visor.

19. The diagnostic device of claim 14, further comprising a pair of handles disposed on sides of the main enclosure.

20. The diagnostic device of claim 19, wherein each of the pair of handles is offset and extends outwardly farther from each of a top surface, a bottom surface, a front surface and a back surface of the main enclosure.

21. A method of diagnosing a subject comprising:
providing diagnostic device including a main enclosure having a subject end and an operator end, at least one infrared light disposed within the main enclosure adjacent the subject end, a stimulus lens, a stimulus screen disposed adjacent the operator end having a front toward a subject and a back, a stimulus filter disposed adjacent the stimulus screen, a mirror, a camera mount disposed adjacent the back of the stimulus screen, a camera coupled to the camera mount and directed toward the mirror, a power source disposed to one side of the main enclosure, a printed circuit board disposed within the main enclosure opposite the power source, and an operator display disposed on the operator end;
holding the diagnostic device so that the operator display faces an operator and the stimulus screen faces the subject; and
providing one or more stimuli on the stimulus screen and presenting results to the operator on the operator display.
